(19) United States
(12) Patent Application Publication
(10) Pub. No.: US 2005/0053668 A1
(43) Pub. Date: Mar. 10, 2005

(54) Skeletally targeted nanoparticles

(75) Inventor: Neal K. Vail, San Antonio, TX (US)

Correspondence Address: FULBRIGHT & JAWORSKI L.L.P., 600 CONGRESS AVE., SUITE 2400, AUSTIN, TX, 78701, US

(73) Assignee: Southwest Research Institute

(21) Appl. No.: 10/922,785

(22) Filed: Aug. 20, 2004

Related U.S. Application Data
(60) Provisional application No. 60/496,740, filed on Aug. 21, 2003.

(51) Int. Cl. A61K 31/66; A61K 9/16; A61K 9/50

(52) U.S. Cl. 424/490; 514/102

(57) ABSTRACT

The invention provides methods and compositions for the delivery of bioactive factors to the systemic skeleton. The methods of the invention allow targeted delivery of bioactive factors to bone using nanocapsules comprised of amphipathic materials. Timed release of bioactive factors may also be used to increase the efficacy of treatment. The methods of the invention have wide applicability for the treatment or prevention of bone-associated maladies.

### BACKGROUND OF THE INVENTION

The present application claims the priority of U.S. provisional patent application Ser. No. 60/496,740, filed Aug. 21, 2003, the entire disclosure of which is incorporated herein by reference.

1. Field of the Invention

The present invention relates generally to the field of medicine. More particularly, it concerns methods and compositions for delivering bioactive factors to bone.

2. Description of Related Art

Numerous pathological conditions are associated with abnormal bone cell function including osteoporosis, osteoarthritis, Paget's disease, osteohalisteresis, osteomalacia, periodontal disease, bone loss resulting from multiple myeloma and other forms of cancer, bone loss resulting from side effects of other medical treatment (such as steroids), and age-related loss of bone mass. Loss of bone mass in particular can lead to skeletal failure such that bone fractures can result from the minimal trauma of everyday life. Such fractures cause significant illness, or morbidity, inasmuch as there is insufficient repair or healing of the fractures.

Osteoporosis is the most common cause of bone loss and is a leading cause of disability in the elderly, particularly in elderly women. Osteoporosis is a progressive disease which results in the reduction of total bone mass and increased bone fragility. This often results in spontaneous fractures of load-bearing bones and the physical and mental deterioration characteristic of immobilizing injuries. The most widely accepted preventive agent for osteoporosis currently in use is estrogen therapy. However, systemic administration of estrogen is not a viable option in women at elevated risk for breast or endometrial cancers (estrogen dependent tumors) or for men (Cooper, 1994). In addition, recent studies have shown that estrogen replacement therapies (ERT's) have other deleterious side-effects, calling into question the long-term effects of these therapies.

Bisphosphonates have been effective inhibitors of osteoclastic bone resorption and have been used to advantage in treating osteoporosis (Parfitt et al., 1996). Bisphosphonates have been shown to increase trabecular bone volume and inhibit the decrease in cancellous bone mass in hindlimb unloaded rats compared to treated controls. However, the amount of cartilage in trabecular bone in these animals significantly increases, indicating that the modeling process is altered and mineralized cartilage fails to be resorbed and replaced by bone.

Vitamin D (1,25D), calcium and thiazide diuretics have also been used alone or in combination to prevent bone loss associated with corticosteroid treatment. The goal of such therapy is to improve calcium absorption and decrease urinary excretion of calcium thus, reversing secondary hyperparathyroidism (Joseph, 1994). Calcium supplements are widely used in managing established osteoporosis but there have been few satisfactory prospective studies of calcium supplementation on bone density or on the risk of future fractures (Cooper, 1994).

Bone damage, such as bone fractures, represents another common bone malady. Although repair, healing and augmentation of bone require a complex series of events that are not well defined, it is known that specific, naturally occurring factors are required to achieve these objectives. Such factors are released or migrate into the injured areas, and stimulate osteoblasts, chondrocytes, and odontoblasts in bone and cartilage to stimulate matrix formation and remodeling of the wounded area (ten Dijke et al., 1989).

New bone can be formed by three basic mechanisms: osteogenesis, osteoconduction and osteoinduction. Cancellous bone and marrow grafts provide viable cells for such processes. Transforming growth factor-beta (TGF-β) has been shown to stimulate proliferation and matrix synthesis of osteoblastic cells (Centrella et al., 1987) and to inhibit the formation and activity of osteoclastic cells (Chenu et al. 1988). Members of the bone morphogenetic protein family have been shown to be useful for induction of cartilage and bone formation. For example, BMP-2 has been shown to be able to induce the formation of new cartilage and/or bone tissue (U.S. Pat. No. 5,013,649). Several small molecules have, such as selected statins (Mundy et al., 1999) and certain proteasome inhibitors (Garrett et al., 2003), have been shown to stimulate BMP-2 production, thus leading to formation of new bone tissue.

Weightlessness during spaceflight has also been a cause of bone loss. Countermeasures for such bone loss have been of the skeletal stress type, such as cycling, simulated running, and rowing (Baldwin et al., 1996). Studies show that exercise-induced skeletal stress serves to maintain and increase osteoblastic activity. However, these methods alone are insufficient to prevent bone volume losses, primarily because it is not possible to generate forces of equal magnitude to those encountered on Earth (McCarthy et al., 2000; Baldwin et al., 1996). Electrical stimulation of selected muscle groups in hindlimb unloaded rats also increases osteoblast activity and osteoid surfaces, but does not prevent decrease in trabecular bone volume or metaphyseal apposition rate (Zerath et al., 1995). Bisphosphonates such as alendronate minimize bone loss during unloading by inhibiting osteoclastic bone resorption, but do not prevent the unloading-induced suppression of bone formation (Bikle et al., 1994). Thus, antiresorbing agents are not ideal countermeasures to bone loss when the primary defect is reduced bone formation (McCarthy et al., 2000).

Growth hormone (GH) treatment of hypophysectomized rats has been shown to increase bone mass independent of whether the animals are loaded or unloaded. However, unloaded animals still show lower bone mass relative to treated animals for the same treatment protocol. Pharmacological doses of GH of 500 μg/ml also failed to mediate skeletal defects in hypophysectomized rats in response to hindlimb unloading, including decreased trabecular bone volume and cortical bone apposition rate (Halloran et al., 1995). Although systemic factors such as GH and 1,25D may modulate the response of bone to unloading, factors that locally regulate bone growth may have greater utility as countermeasure molecules to prevent bone loss.

While the above described countermeasures to bone loss have been successful in minimizing to some extent the morbidity associated with abnormal bone cell function, the efficacy of such treatments is limited by the ability to appropriately deliver the active ingredient to the site where needed. In addition, most of these treatments have serious side effects when administered systemically.

Therefore, the need for site specific targeting of therapeutic agents has been felt. However, site-specific targeting requires quantitatively distinct receptors that are unique to bone. The inorganic component of bone which is comprised of hydroxyapatite (HAp), occurs normally only in hard tissues. Bisphosphonates, such as methylene bisphosphonate (MBP) and others, are known for their predilection to bone sites undergoing remodeling. In particular, MBP has been used in combination with Technetium-99m (^{99m}Tc) as a non-therapeutic diagnostic imaging tool in the study of bone pathology (Davis and Jones, 1976; Lantto et al., 1987; Cronhjort et al., 1999). Therapeutic analogs of MBP, including alendronate, risedronate, and others, exploit the well-known ability of the bisphosphonate scaffold to bind strongly to the surface of solid-phase calcium phosphate as the basis of their action on bone resorption (Fleisch, 1995). MBP has been studied as a bone matrix targeting moiety for osteotropic drug delivery. Fujisaki, et al., (1995; 1996), conjugated various model materials and pro-drug candidates to MBP and demonstrated their targeting efficacy in vivo. Estradiol conjugated to MBP was taken up in bone and then released from MBP either by enzymatic or chemical hydrolysis of the ester conjugation linkage. Uludag, et al., (2000a; 2000b), demonstrated the osteotropic delivery of model proteins conjugated to MBP by similar chemistry.

In addition, several bone non-collagenous proteins, such as osteopontin and bone sialoprotein, are known to contain amino residue sequences that bind specifically to HAp (Nagata et al., 1991). Fujisawa et al., determined that a six-residue aspartic acid oligopeptide (Asp₆) preferentially binds to the calcified matrix in vivo (Kasugai et al., 2000) and that this targeting ligand can deliver an estradiol pro-drug in vivo (Yokogawa et al., 2000).

Prior approaches for targeting bone that use simple molecules conjugated to bone-targeting ligands that preferentially accumulated in bone have serious drawbacks. First, conjugated molecules are systemically exposed and, therefore, are subject to rapid elimination or can have action on sites other than bone. Second, conjugation of the bone-targeting ligands to the therapeutic molecules can adversely alter their therapeutic activity. Lastly, release of the active molecule from the targeting ligand, and its subsequent activity, is dependent on the degradation kinetics of the conjugation linkage.

Nonetheless, the aging global population translates to ever-increasing demand for orthopedic countermeasures to skeletal deterioration resulting from the increasing fragility of skeletal structures with age. In addition, there is an acute need to find effective countermeasures for other bone conditions and ailments. There is, therefore, a great need in the art for novel therapeutic compositions that can be used to deliver therapeutic agents to targets in the bone for effective treatment of bone-associated maladies.

### SUMMARY OF THE INVENTION

In certain aspects, the invention provides enhanced techniques for bone-targeting comprising use of nanocapsule delivery vehicles. Encapsulation provides an opportunity to consider therapeutics that are efficacious in treating bone diseases, but that otherwise may be rapidly cleared from the system, only elicit a biological response in close proximity to target cells, be toxic to other cell phenotypes, or be systemically active. In certain aspects of the invention, the targeted delivery of therapeutic agents to the bone is extended by including the capabilities of controlled and triggered release of the therapeutic agents either through engineered degradation of the delivery vehicle or in response to localized stimuli.

In one aspect, the invention provides a nanocapsule comprised of an amphipathic surface encapsulating at least a first hydrophobic bioactive factor, wherein the surface comprises at least a first ligand having affinity for a component of the systemic skeleton. The nanocapsule may be defined as a micellar nanocapsule. In one embodiment, the ligand has affinity for hydroxyapatite in said systemic skeleton. In particular embodiments, there may be a bisphosphonate comprising the structure: 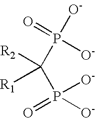
wherein R1 is H, OH or, Cl and wherein R2 is an alkyl amine or other heterobifunctional linker coupled to the nanocapsule surface. The ligand may also be amino methylene bisphosphonate (aMBP), and may be a protein, a peptide an oligopeptide, or an antibody, including an oligopeptide comprising the sequence Aspₙ, for example, wherein n=6. Examples of such a protein or peptide comprise the sequence of osteopontin or bone sialoprotein or a fragment thereof.

In certain embodiments of the invention, a nanocapsule provided may have a diameter of from about 1 nm to 200 nm, including about 10 nm to about 50 mm, about 50 nm to about 100 nm, about 100 nm to about 150 nm, and about 150 m to about 200 nm. The first target ligand may be covalently bound to said surface. The bioactive factor may be an osteotropic agent or an anabolic agent. The amphipathic surface may be comprised of a block polymer and may be comprised of polyethylene glycol-modified phospholipid. An example of such a material is distearyol phosphoethanolamine-N-polyethylene glycol (DSPE-PEG). In certain embodiments, the amphipathic surface is comprised of a material with a critical micelle concentration of between about 1 μm and about 20 μm.

In another aspect, the invention provides a method of delivering a bioactive factor to a component of the systemic skeleton of a patient in need thereof comprising: (a) obtaining nanocapsules comprised of an amphipathic surface encapsulating at least a first hydrophobic bioactive factor, wherein the surface comprises at least a first ligand having affinity for a component of the systemic skeleton; and (b) administering the composition to the patient. In the method the nanocapsules may be micellar nanocapsules. The ligand may have an affinity for hydroxyapatite in said systemic skeleton, and may comprise amino methylene bisphosphonate (aMBP) or an oligopeptide such as Aspₙ. The composition may comprise a pharmaceutically acceptable carrier and the bioactive factor may be released from said nanocapsules upon contact of said nanocapsule with a signal released from said systemic skeleton. The nanocapsules may comprise varied temporal release characteristics. Such nanocapsules may a have a diameter of from about 1 run to 200 nm, including about 50 nm to about 100 nm, about 100 nm to about 150 nm, and about 150 nm to about 200 nm, as described herein. The bioactive factor may be selected from the group consisting of a statin, a proteasome inhibitor, and an antiosteoporotic alkyloid and may be an osteotropic agent, hormonally active or an anabolic agent. The amphipathic surface may be comprised of a block polymer, and may be a polyethylene glycol-modified phospholipid, such as phosphoethanolamine-N-polyethylene glycol (DSPE-PEG). The amphipathic surface may be comprised of a material with a critical micelle concentration of between about 1 μm and about 20 μm. The composition may be delivered by any desired means including locally, systemically, intravenously, intra-arterially, topically or orally.

In still yet another aspect, the invention provides a method of preventing bone loss in a subject in need thereof comprising: (a) obtaining nanocapsules comprised of an amphipathic surface encapsulating at least a first hydrophobic osteotropic factor, wherein the surface comprises at least a first ligand having affinity for a component of the systemic skeleton; and (b) administering the composition to the patient. In the method the nanocapsules may be micellar. The ligand may have an affinity for hydroxyapatite in said systemic skeleton. In certain embodiments the osteotropic factor is released from said nanocapsules upon contact of said nanocapsules with a signal released from said systemic skeleton. The nanocapsules may comprise varied release characteristics. Examples of suitable ligands include aMBP and Aspₙ. The composition may be formulated in a pharmaceutically acceptable carrier and may be delivered in any desired manner such as locally, intranasally, systemically, intravenously, intra-arterially, topically or orally.

In one aspect of the invention, the range of content of bioactive ingredient in the nanocapsules of the invention is in the order of about 0.01-8 mol %. Thus, one may use about 0.01 mol %, 0.02 mol %, 0.05 mol %, 0.1 mol %, 0.25 mol %, 0.5 mol %, 1.0 mol %, 2.0 mol %, 2.5 mol %, 3.0 mol %, 4. mol %, 5.0 mol %, 6.0 mol %, 7.0 mol % and about 8.0 mol % of the targeting ligand in a nanocapsule. All intermediate ranges of such mounts are specifically contemplated.

In specific aspects of the invention, the nanocapsule has a diameter of from 1 nm to 200 nm. In non-limiting embodiments of the invention, the nanocapsule has a diameter of about 50 nm, about 100 nm, about 150 nm, or about 200 mm. It is contemplated that the nanocapsules of the invention may have diameters of about 10 nm, 20 nm, 30 nm, 40 nm, 60 nm, 70 nm, 80 nm, 90 nm, 110 mm, 120 nm, 130 nm, 140 nm, 160 nm, 170 nm, 180 nm, 190 nm as well as intermediates such as 5 nm, 15 nm, 17 nm, 38 nm, 240 nm and the like.

In some aspects of the invention, the nanocapsules may comprise a bioactive factor that can prevent or treat any bone-related disorder or condition. The use of any bioactive factor known in the art to treat a bone-related condition may be used. In some embodiments of this aspect of the invention, the bioactive factor is a bone morphogenetic protein, a proteasome inhibitor, a protein fragment, a peptide, estrogen, a bisphosphonate, TGF-β, an antiosteoporotic alkaloid, a non-peptide small molecule, and an osteotropic agent. In other embodiments of the invention, the bioactive factor is an osteotropic agent. In still other embodiments of the invention, the bioactive factor is a peptide. In specific aspects of this embodiment of the invention, the peptide is hormonally active. In yet other embodiments of the invention, the bioactive factor is a protein. In still other embodiments of the invention, the bioactive factor is a non-peptide small molecule.

In one aspect of the invention, the patient or subject to whom the composition is administered is one who is afflicted by a bone related disorder or condition. In other aspects, the patient or subject can be one who is at a risk of developing a bone related disorder or condition and therefore the method will be a preventive method. Some non-limiting examples of bone-disorders and conditions afflicting patients or subjects that are treatable or preventable by the methods of the present invention include osteoporosis, osteoarthritis, Paget's disease, osteohalisteresis, osteomalacia, periodontal disease, multiple myeloma and other metastatic bone cancers, bone loss resulting from multiple myeloma and other forms of cancer, bone loss steroid treatments, or bone fractures, and age-related loss of bone mass.

Different methods of administering therapeutic compositions are known in the art and any such method may be used to deliver the therapeutic compositions of the present invention. In some non-limiting embodiments of the invention, the compositions may be administered locally, systemically or regionally. In other non-limiting embodiments of the invention, the compositions may be administered intravenously, intra-arterially, topically, orally, or nasally. In still other embodiments of the invention, other methods of parenteral administration, such as muscular, sub-cutaneous, intraperitoneal, intralesionally, dermally, are also contemplated.

“A” or “an” is defined herein to mean one or more than one. Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A, 1B, & 1C. FIG. 1A: Targeting nanocapsules are introduced to the system by an appropriate means. The nanocapsules are comprised of a membrane encompassing, containing, or stabilizing a therapeutic payload. The nanocapsule membrane is covered with targeting ligands that will selectively bind to targeted sites located in bone. FIG. 1B. Targeting nanocapsules selectively bind to the hydroxyapatite component of the bone matrix via targeting ligands. FIG. 1C. The therapeutic payload is released following targeted site attachment.

FIG. 2. Older population by age: 1900-2050 for the age groups 65+ and 86+ years of age (source: US Department of Health and Human Services, Administration on Aging).

FIG. 3. Synthesis pathways to attach targeting-ligands to phospholipids or PEGylated phospholipids.

FIGS. 4A & 4B. FIG. 4A. Evolution of liposome particle size with decreasing extrusion pore size. Liposomes were extruded twice through 2 μm pores, four times through 0.4 μm pores and eight time through 0.1 μm pores. FIG. 4B Typical final liposome particle size distribution trace. Liposome size is 112.1 nm ±μ23.8 nm.

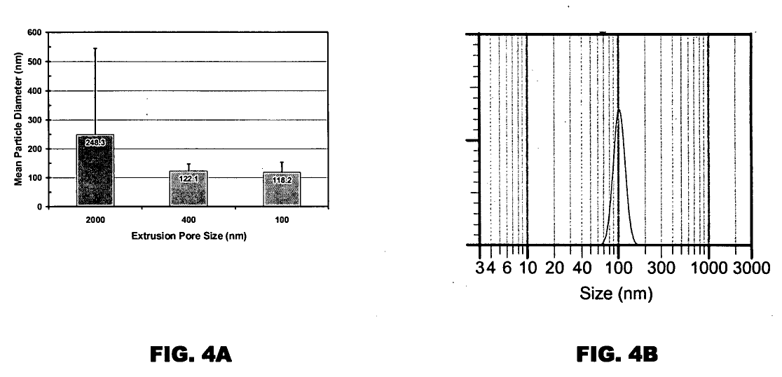

FIG. 5. Example of phospholipid conjugation strategy to attach MBP to a phospholipid. This strategy is the same for Asp₆ oligopeptide, in which conjugation is done through the N-terminus.

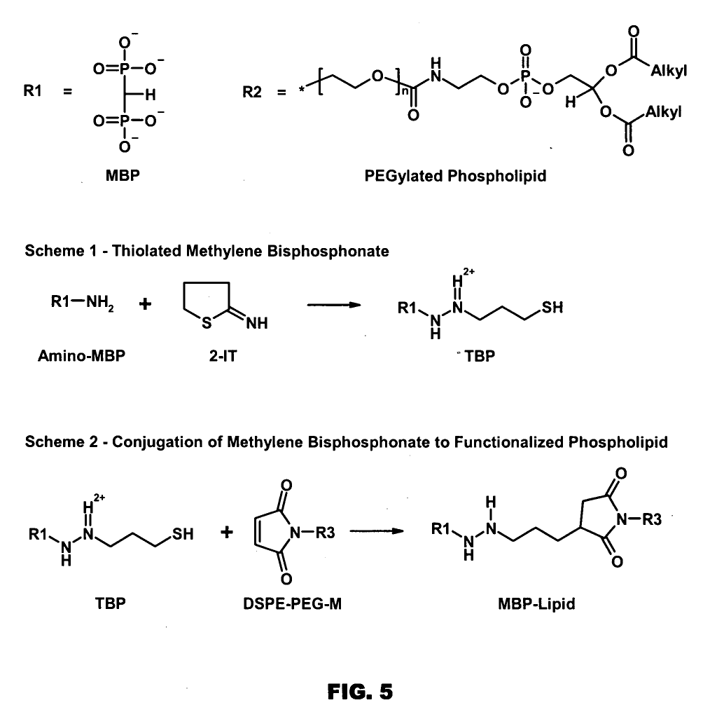

FIG. 6. A matrix-assisted laser desorption ionization time-of-flight/mass spectroscopy spectrum of a ligand-phospholipid conjugate. The ligand is methylene bisphosphonate and has a molecular mass of 280. The phospholipid is distearoyl phosphotidylethanolamine-N-methoxypolyethylene glycol-2000 and has a reported molecular mass of 3037 (measured 3010 by MALDI TOF/MS). The conjugate has a median mass of 3318 (theoretical mass is 3331).

FIG. 7. Surface area normalized adsorption of FITC-labeled ligands onto various hydroxyapatite substrates as a function of the amount of labeled ligand charged to the system. The aspartic acid oligomer adsorbs slightly better to the HAp surface than the methylene bisphosphonate ligand.

FIG. 8. Absolute adsorption of methylene bisphosphonate-containing liposomes onto various hydroxyapatite substrates. The HA1 substrate has a higher surface area than the HA2 substrate. The HA2 substrate is quickly saturated while the HA1 continues to adsorb targeting nanocapsules.

FIG. 9. Distribution of lovastatin in micelles prepared from the methylene bisphosponate-based ligand-phospholipid conjugate. The micelles encapsulate up to about 0.03 μg of lovastatin/μg of lipid before free drug precipitates from the system.

### DESCRIPTION OF ILLUSTRATIVE EMBODIMENTS

The invention overcomes the deficiencies of the prior art by providing nanocapsules for the targeted delivery of therapeutic agents to the systemic skeleton. The compositions and methods of the invention can be used for the delivery of potentially any bioactive factor to bone. The bioactive factors are delivered in nanocapsules comprising a payload of the bioactive factor and that are targeted for specific delivery to bone. The nanocapsules can be delivered non-invasively as a therapeutic or as a countermeasure designed to prevent development of bone abnormalities. The invention has application for a variety of conditions associated with bone loss, bone abnormalities or bone damage including, but not limited to osteoporosis, osteoarthritis, Paget's disease, osteohalisteresis, osteomalacia, periodontal disease, myeloma and other metastatic bone cancers, bone loss resulting from multiple myeloma and other forms of cancer, bone loss resulting from side effects of other medical treatment (such as steroids), bone loss resulting from fractures, prosthesis fixation and age-related or weightlessness-related loss of bone mass. Anabolic agents in particular are not selective to bone and their therapeutic-toxic window may be narrow hence, the need for targeted delivery mechanisms cannot be overstated.

The invention provides in certain aspects micellar nanocapsules targeted to the systemic skeleton. By forming the micellar nanocapsules from amphipathic materials such as polyethylene glycol-modified phospholipids and other block-type polymers bound to targeting agents for the systemic skeleton, the invention allows preferential targeting of bone with encapsulated hydrophobic biological agents. The invention represents a significant advance over liposome-based nanocapsules for delivery of hydrophobic drugs. Liposomes primarily comprise cell-like vesicles formed from or encompassing phospholipid bilayers. The bilayers consist of two mutually oriented layers of amphipathic molecules that orient to produce a barrier between an exterior continuous aqueous phase and the entrapped aqueous volume. The bilayers are essentially an organic oil that may have other lipophilic additives to impart stability or provide selective bilayer transport of molecules species.

Due to the structure of liposomes they are primarily suitable for the encapsulation of hydrophilic species within the entrapped aqueous volume. Encapsulation of hydrophobic species is possible by making use of water-soluble complexes or by incorporating the hydrophobic species into the bilayer membrane, but this has inherent downsides. In the case of water-soluble complexes, the hydrophobic species is complexed with the water-soluble host molecule, for example, a cyclodextrin, that can accommodate the hydrophobe as a guest molecule. The host-guest complex can then be encapsulated in the aqueous interior of the liposome. The downsides to this approach include low complexation efficiencies, increased temperature processing to affect host-guest complexing, and guest exchange of the complex with lipid bilayer constituents (e.g., cholesterol). In the bilayer inclusion case, the amount of uptake of the hydrophobe by the bilayer will be limited by molecule size and bilayer composition. The downsides will be low encapsulation efficiency and rapid bilayer exchange at physiologic conditions.

The invention overcomes these limitations in one embodiment by providing micellar nanocapsules comprised of amphipathic materials to achieve efficient encapsulation of hydrophobic bioactive agents. The amphipathic materials may be modified with site-specific targeting ligands. In aqueous systems containing these materials at concentrations at or above the so-called critical micelle concentration (CMC), the amphipathic materials will preferentially form micelles having a hydrophobic interior and hydrophilic exterior. The hydrophobic interior is ideal for the solubilization of hydrophobic molecules. If amphipathic materials with low CMCs are used (for example, 1-20 μM), the resulting micelles will be extremely stable at low concentrations and suitable for drug applications under physiological conditions. Further, if the hydrophilic end of the amphipathic materials is functionalized with a ligand having specifically for selected tissue site, preferential site targeting and drug delivery occurs.

Natural and synthetic phospholipids, PEG-modified diacyl phospholipids and AB or ABA block-type polymers in particular will find use for the production of micellar nanoparticles in accordance with the invention. An example of a PEG-modified phospholipid is distearyol phosphoethanolamine-N-polyethylene glycol (DSPE-PEG). This material can be efficiently functionalized with ligands, such as methylene bisphosphate or aspartic acid oligomers, which have demonstrable binding affinity for the calcified matrix of bone (calcium phosphate), to achieve bone-targeting capabilities. DSPE-PEG and its analogues, have extremely low CMCs (typically 1-20 μM), and produce stable micelles in buffered systems. Therefore, the invention provides methods of preferentially delivering an active factor to the musculoskeletal system in nanocapsules comprised of an amphipathic material having a sufficiently low critical micelle concentration to achieve micelle stability at physiologic conditions. Similarly, block copolymers comprised of discrete hydrophobic and hydrophilic blocks can be used to form stabilized hydrophobic phases. Phase morphology, size, and stability can be selected by adjusting the relative block lengths of the copolymer. A particular example of this type of material is polyethylene oxide-co-polyethylpropylene oxide block copolymers, such as those in the Pluronic and the Poloxamer series. Other examples of amphipathic block copolymers include polyethylene oxide-co-polycaprolactone, polyethylene oxide-co-aspartates, polyacrylic acid-co-polystyrene, polyethylene oxide-co-polybutadiene, polyethylene oxide-co-polyethylethylene, polyethylene oxide-co-polylactic acid, polydimethylsiloxane-co-poly(2-methyloxazoline), and polyethylethylene-co-polystyrene sulfonic acid, etc.

By allowing targeted delivery of bioactive factors, the invention provides preferential delivery of the bioactive factors at the skeletal site where the factors are needed, e.g., to the affected bone matrix. Localized delivery increases the potency without an increase in the dosage, and also reduces side-effects incurred by systemic exposure. The nanocapsules of the invention can also be designed for controlled or triggered release of payloads, for example, by capsule degradation and payload diffusion or in response to external signals or physiologic signals occurring in the bone microenvironment. This allows therapeutic release specifically to sites where and when treatment is needed. Targeted and/or timed delivery of bioactive factors also allows administration of a lower overall dose of the bioactive factor to the patient, minimizing the potential for adverse side effects due to narrow therapeutic-toxic windows. Methods and compositions for such delivery and targeting of nanocapsules are discussed in U.S. patent application Ser. No. 10/350,805, filed Jan. 24, 2003, the entire disclosure of which is specifically incorporated herein by reference.

The nanocapsule drug vehicles can be targeted by fabricating the nanocapsules to contain both surface-bound bone-specific targeting ligands and payloads comprising bioactive therapeutic agents, compounds or countermeasures. Such surface-bound bone targeting ligands can specifically target the bone mineral phase. Examples of targeting ligands that may be used include bisphosphonates and oligopeptides, which have been shown to preferentially bind to bone.

Targeting to bone can be achieved in particular by surface-functionalizing nanocapsules with hydroxyapatite (HAp) binding residues (e.g., bisphosphonates, peptide residues, etc.). Therefore, the nanocapsules can be comprised of targeting ligands, a membrane component and a therapeutic payload. The targeting ligands can be attached to a nanocapsule membrane and can selectively bind to targeted sites within the systemic skeleton.

One application of the invention is in the delivery of bioactive factors to maintain skeletal health by preventing bone loss. Such factors may prevent bone resorption, for example, to treat osteoporosis or as a maintenance program for the prophylactic prevention of bone loss. Such prophylactic treatment may find use, for example, in preventing bone loss during manned spaceflight. Bone mass loss is also a growing problem for the rapidly aging population, and could be prevented by administration of the targeted nanocapsules provided by the invention. One such bioactive factor that may be used is transforming growth factor beta (TGF-β), which activates cell proliferation and metabolic pathways in osteoblast-like cells in vitro. Other non-limiting examples of bioactive peptides that may be used are other sub-classes of the TGF-β family of peptides and the bone morphogenetic proteins. Still other bioactive factors that may be used are compounds that stimulate expression of bone morphogenetic protein 2. Other non-limiting examples of such BMP-2 expression stimulators are certain statins and proteasome inhibitors. All these molecules are well known in the art.

Specific targeting of nanocapsules also allows targeting of skeletal structures, especially areas of high bone turnover (e.g., areas undergoing active resorption due to disease or non-loaded use). The targeted nanocapsules can also be used to deliver other selected therapeutics to bone, including, for example, treatment of infection or tumors in bone via antibiotics or cancer therapies, respectively. Similarly, the nanocapsules may also find use of fracture repair therapies and tissue engineering applications.

I. Targeted Delivery of Bioactive Factors

A. Countermeasure Methods

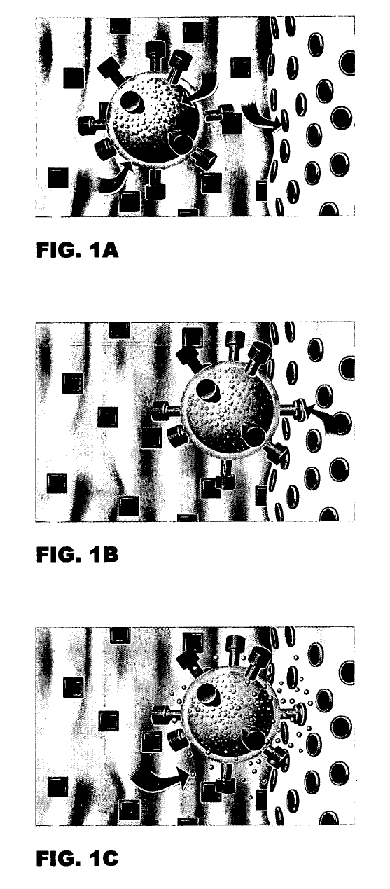

One aspect of the invention is set forth, for illustrative purposes only, in FIG. 1. In this approach, a bioactive factor is incorporated into a nanocapsule vehicle in accordance with the invention. The nanocapsule is designed to specifically target bone, for example, by targeting the hydroxyapatite (HAp) component of the skeletal matrix. In vivo, the targeted nanocapsules preferentially bind to exposed HAp surfaces (e.g., especially bone matrix locations undergoing osteoclastic resorption), whereupon they subsequently disrupt to release the therapeutic payload contained in the nanocapsules.

In a second approach, the nanocapsules may be designed to release their therapeutic payload temporally in response to a specific stimulus. This stimulus could be an externally applied signal, a complementary factor administered in schedule, or may be a biochemical signal present in the bone microenvironment. In this way, delivery can be made at locations where and when needed or otherwise appropriate. If the nanocapsules are not exposed to the appropriate signal or factor, the nanocapsules remain intact and are eventually expelled by the body through normal metabolic activities. Therefore, any side effects associated with the bioactive factor(s) contained in the nanocapsules may be avoided when treatment is not needed. Still further, lower effective doses of the bioactive factor in the locally affected bone microenvironment will be received by the patient when the treatment is needed.

The bioactive factor could be one or more of many agents that have been shown to impact the formation of new bone matrix (e.g., BMPs, protein fragments, statins, proteasome inhibitors, estrogens, molecular conjugates, etc.) or to have any other desired therapeutic or preventative effect with respect to any bone-associated malady. As indicated, the bioactive factors may be encapsulated in micellar nanocapsules according to the invention that may be designed to have controlled temporal release in the appropriate physiological environment. Some examples of bioactive factors that could be delivered with the invention include insulin-like growth factors (IGF), bone morphogenetic proteins (BMP), heparin-binding fibroblast growth factor (FGF), platelet-derived growth factors (PDGF), TGF-β, parathyroid hormone (PTH), fluoride, statins, antiosteoporotic alkoloids, and proteasome inhibitors.

The nanocapsules could be introduced systemically by intravenous injection or non-invasively by intranasal uptake, or topical application. In vivo, the nanocapsules would preferentially bind to exposed HAp surfaces (e.g., especially bone matrix locations undergoing osteoclastic resorption), whereupon they would subsequently disrupt to release their therapeutic payload.

B. Bone Loss

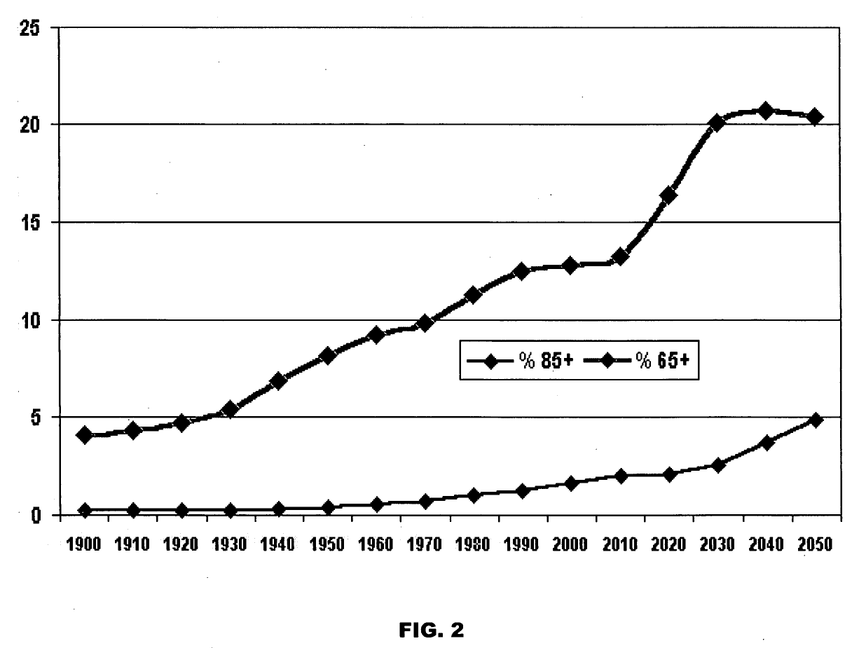

As described above, one application of the methods of the invention is in the prevention or treatment of bone loss. The aging global population translates to ever-increasing demand for such countermeasures to skeletal deterioration resulting from the increasing fragility of skeletal structures with age. Demographic trends in the United States, Europe and Japan are similar, with the percentage of the population over the age of 65 increasing dramatically (FIG. 2) (see, e.g., US Department of Health and Human Services, Administration on Aging, www.aoa.dhhs.gov). This underscores the importance of the invention. Maintaining or restoring bone volume is therefore a major health care issue for an increasingly vast segment of the general population (see, e.g., Trends in Orthopedics, 2000; Orthopedic Industry, 2000). The need for a therapeutic protocol that targets the systemic skeleton to maintain and/or increase bone volume cannot be overstated.

Yet another application of this technology is with regard to fracture healing and prevention of bone loss during fractures. Targeted delivery of bone healing agents, such as anabolic agents, to the bone can minimize the time of bone-healing and also prevent loss of existing bone tissue. In addition, the invention is contemplated useful in the process of prosthetic fixation.

Another possible source of bone loss which could be treated with the invention is spaceflight. Astronaut health during long-term space flight is a major concern and central to this general health concern are the effects of space flight on skeletal tissues. Skeletal degradation can dramatically affect the ability to perform both rudimentary tasks and critical extravehicular activities during long-term space missions. Astronauts experience a 1-2% decrease in bone volume per month at selected skeletal sites and this bone loss is generally not fully recovered on return to Earth (National Geographic, January, 2001; Vico et al., 2000). This rate of bone volume loss could cause decreases in bone mineral density (BMD) of more than 50% during a 2-3 year Mars mission, significantly impairing an astronaut's abilities during space flight and on entry into gravitational environments. The consensus is that bone loss countermeasures are necessary for continued space exploration.

Knowledge of human adaptation to long-term space flight lags behind the technical knowledge required for such travel (Turner, 2000). Experience in near-earth space flight suggests that most biological effects on the skeletal system result from changes in physical loading of the skeleton. This stems from the fact that an astronaut in near-earth orbit, though still within the gravitational field, experiences free-fall as an adjunct of spacecraft velocity. Changes in bone biology and the associated loss in bone volume begin to occur within a few days after leaving Earth. MIR space station studies clearly show that individuals experience decreases in BMD in load-bearing areas such as the lumbar spine, proximal femur, and calcaneus, while non-load-bearing areas, such as the cranium, distal radius, and ribs, experience increases in BMD (McCarthy et al., 2000). Experiments have shown a decrease in the expression of selected bone matrix cytokines (Carmeliet et al., 1998), such as TGF-β and insulin-like growth factor-i (IGF-1), both of which are known to regulate bone formation (Mundy, 1996). Similar findings have been reported in cell culture studies wherein osteoblast activity and associated deposition of new skeletal matrix both decrease. Thus, it is clear that the normal bone remodeling process is profoundly altered during space flight. Osteoblastic bone formation decreases and osteoclastic resorption activity either remains unchanged or slightly increases. The net result is the onset of osteopenia (Holick, 2000).

Infusion of IGF-1 stimulates bone growth in normally loaded bones but in unloaded bones, an extremely high dose of 2 mg/kg/day is required to demonstrate any protective effect against unloading (Bikle et al., 1994). Decreases in TGF-β message levels have been observed in three different models of skeletal unloading: spaceflight, sciatic neurotomy, and hindlimb unloading (Westerlind and Turner, 1995). These results are consistent with a growing body of evidence suggesting that reduced bone formation during spaceflight is due to decreased osteoblast function (Harris et al., 2000). Significantly, infusion of TGF-β (2 μg/kg/day) corrects the decrease in bone mass, calcium content, osteoblast number and mineralization rate induced by hindlimb unloading in rats, but has no effect on bone formation in control animals. Further, TGF-β infusion decreases the indices of bone resorption in both normal and unloaded rats (Machwate et al., 1995). Effective targeting and delivery of TGF-β to increased concentrations locally in the bone microenvironment would be a desirable goal of any drug delivery countermeasure (Mundy, 2000).

Still a further application of this technology is in the treatment of multiple myeloma and other metastatic bone cancers. Multiple myeloma, a clonal hematopoietic neoplasm of plasma cells, is the second most common adult hematologic malignancy and is unique among hematologic neoplasms in its propensity to cause bone destruction (Mundy, 1998). It has a worldwide prevalence of about 145,000 cases (Parkin et al., 1999), affects 70,000 Americans, accruing 15,000 new cases yearly, and accounting for 1-2% of cancer-related deaths (Jemel et al., 2003, Kyle et al., 2003). The disease is uniformly fatal with 80% of patients suffering devastating and progressive bone destruction. The high morbidity and mortality associated with this malignancy are attributable to the consequences of bone destruction, such as excruciating and unremitting bone pain, pathologic fractures, nerve compression syndromes such as spinal cord compression, and life-threatening hypercalcemia. In most patients, multiple discrete lytic lesions occur adjacent to nests of myeloma cells, but occasionally patients present with diffuse osteopenia simultaneously occurring in multiple bones in both the axial and appendicular skeleton.

Beneficial effects of conventional therapeutic regimens are modest and relapse is invariable. Specific treatment of the disease with bisphosphonate inhibitors of bone resorption reduces osteolytic lesions in animal models and in myeloma patients, but does not reduce total myeloma cell burden and further has not been shown to prolong survival. The average life span after diagnosis is less than three years, a statistic that has not changed significantly in thirty years (Bataille et al., 1997). Reductions in myeloma bone 25442332.1 disease that translate into improved clinical prognosis are likely most effective only with agents that also control tumor growth and progression. Because of the severe morbidity associated with myeloma bone disease and because myeloma cannot be cured by presently available chemotherapy regimens or stem cell transplantation, new treatment strategies are of urgent and vital importance.

A recent major development in the clinical management of cancer patients has been targeting of the 26S proteasome as an anticancer modality (Jemel et al., 2003; Parkin et al., 1999). Inhibition of proteasomal function has emerged as a novel therapeutic approach with substantial and remarkable antitumor effects in patients. This is exemplified by the discovery and development of the antitumor agent, PS-341, a boronated dipeptide that works by reversibly inhibiting proteasome function (Adams, 2002). PS-341 was originally shown to be cytotoxic against a panel of tumor cells. The drug is a potent inhibitor of both myeloma cell growth and survival in vitro. Significantly, there are critical and unresolved issues in the use of PS-341, and other proteasome inhibitors, as therapeutic modalities in myeloma.

Clinical use of PS-341 has been associated with many side-effects, such as thrombocytopenia, peripheral neuropathy, hypotension, and, in severe cases, cardiovascular toxicity. Interestingly, however, it has been shown that several structurally-unrelated inhibitors that bind to specific catalytic β subunits of the 20S proteasome stimulated bone formation in bone organ cultures in concentrations as low as 10 nM, a far more potent effect than observed for other small molecules, suggesting that this class compounds could have synergistic effect on the inducement of new bone growth (Garrett et al., 2003). Therefore, it is important to maximize the efficacy of these compounds while reducing deleterious systemic side-effects. This is best achieved using targeted drug delivery mechanisms to facilitate uptake of the drug at the site of interest, in this case, to lesions in bone, which may be carried out according to the invention.

C. Targeted Delivery of Nanocapsules

Targeted delivery of therapeutics via nanocapsules can occur by either passive or active mechanisms. Passive targeting occurs when nanocapsules extravasate through damaged vasculature to accumulate in tumors and inflamed tissues (Wu et al., 1993). Accumulation increases by improving circulation half-life and by preventing nanocapsules interaction with serum components. In contrast, active targeting is achieved through specific interaction between nanocapsule-bound or -associated ligands and complementary binding agents at the targeted site. This approach has clear opportunity for improved, efficacious delivery of bioactive agents, since many do not target bone, have narrow therapeutic-toxic windows when administered systemically, and require close proximity to target cells to exert their biological activity.

Prior techniques employing liposome-based targeting approaches have used one of a handful of methods, including receptor targeting, cell adhesion molecules, extracellular matrix molecules, selecting, and antibody ligands (Forssen and Willis, 1998). Lee and Huang (1995) demonstrated a 45-fold increase in doxorubicin uptake from folate-modified liposomes in to epithelial cancer cells, which over-express folate receptors. Kamps, et al. (1997), demonstrated that liposomes modified with anionized albumin were taken up by hepatic endothelial cells, whereas nearly all non-modified liposomes remained in circulation 30 minutes post-injection.

Bone offers several potential sites for targeted delivery of bioactive agents. Bone is a composite matrix comprised of organic and inorganic constituents. The organic portion of the matrix consists of a mixture of collagen, bone proteins, water, and cells (Rho et al., 1998). The inorganic portion of the matrix consists chiefly of hydroxyapatite (HAp). While it is possible to selectively bind bioactive constituents to specific receptors located either on bone cells or on bone proteins, such targeting may not be sufficiently specific. Similar receptors may exist on other cell phenotypes and many bone proteins can be found external to the bone matrix. Site-specific targeting requires targeting receptors quantitatively distinct from receptor sites found in other tissues. For this reason, HAp, which occurs normally only in hard tissues, provides one attractive targeting site for the selective delivery of bioactive agents to bone.

1. Targeting Ligands

In certain aspects of the invention, ligands having an affinity with bone are linked to nanocapsules. Two ligands in particular that may be used for targeting of nanocapsules to, for example, the HAp portion of the bone matrix without an associated therapeutic effect are methylene bisphosphonate (MBP) and an aspartic acid peptide residue. MBP is well known for its predilection to bone remodeling sites. For this reason, it has been used extensively in combination with Technetium-99m (^{99m}Tc) as a diagnostic imaging tool in the study of bone pathology (Davis and Jones, 1976; Lantto et al., 1987; Cronhjort et al., 1999). MBP has further been studied as a bone matrix-targeting moiety for osteotropic drug delivery. Fujisaki, et al., (1995 and 1996) have conjugated various model materials and prodrug candidates to MBP and demonstrated their efficacy in vivo. Estradiol conjugated to MBP has been shown to be rapidly taken up in bone and then to be released from MBP either by enzymatic or chemical hydrolysis of the ester conjugation linkage. Uludag, et al. (2000), have demonstrated the osteotropic delivery of model proteins conjugated to MBP by similar chemistry. The chemical formula for MBP is given below:

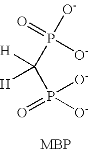

Several bone noncollagenous proteins, such as osteopontin and bone sialoprotein, are also known to contain amino residue sequences that bind specifically to HAp. For example, Fujisawa, et al., determined that a six-residue aspartic acid oligopeptide (Asp₆) would preferentially bind to the calcified matrix in vivo (Kasugai et al., 2000). They further showed that this targeting ligand could deliver an estradiol prodrug in vivo (Yokogawa et al., 2000). The chemical formula of this targeting ligand is given below: 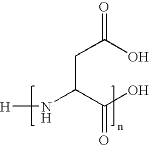 (Asp)ₙ=4−6

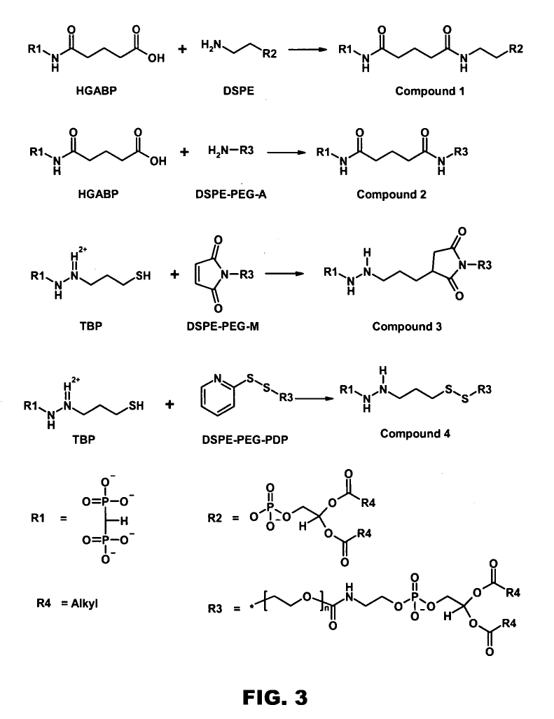

Yet another ligand that could be used is 1-amino-1,1-diphosphonate methane (ABP). The amino functionalized analogue of methylene diphosphonate can be synthesized by published methods (Kontoci et al, 1996) as modified by Uludag, et al (2000). Once synthesized, the molecular structure of ABP can be confirmed by 1H, 13C, and 31P NMR. A description of the technique for synthesis of ABP-phospholipid and ABP-PEGylated phospholipid conjugates is set forth in FIG. 3. Still another ligand that can be used is alendronic acid.

2. Linking Targeting Ligands to Nanocapsules

Bifunctional cross-linking reagents represent one means for attaching a targeting ligand to a nanocapsule that may be used with the invention. Bifunctional cross-linking reagents have been extensively used for a variety of purposes including preparation of affinity matrices, modification and stabilization of diverse structures, identification of ligand and receptor binding sites, and structural studies and can be used for linking targeting agents to nanocapsules. Homobifunctional reagents that carry two identical functional groups have proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to nanocapsules are described in U.S. Pat. No. 5,603,872 and U.S. Pat. No. 5,401,511, each specifically incorporated herein by reference in its entirety. Various ligands can be covalently bound to a nanocapsule surface through the cross-linking of amine residues. The inclusion of a PEGylated phosphatidylethanolamine (PE) in a nanocapsule according to the invention provides an active functional residue, a primary amine, on the surface for cross-linking purposes.

Ligands can be bound covalently to discrete sites on nanocapsule surfaces. The number and surface density of these sites will be dictated by the nanocapsule type. The nanocapsule surfaces may also have sites for non-covalent association. To form covalent conjugates of ligands and nanocapsule, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and nanocapsules is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups and is thus useful for cross-linking polypeptides and sugars. Table 1 details examples of certain hetero-bifunctional cross-linkers that may be used in accordance with the invention.

TABLE 1
Hetero-Bifunctional Cross-Linkers

| | | | Spacer |
|---|---|---|---|
| | | | Arm Length\ |
| | | Advantages and | after |
| Linker | Reactive Toward | Applications | cross-linking |
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC- | Primary amines | Extended spacer arm | 15.6 A |
| SPDP | Sulfhydryls | Water-soluble | |
| SMCC | Primary amines | Stable maleimide reactive | 11.6 A |
| | Sulfhydryls | group | |
| | | Enzyme-antibody | |
| | | conjugation | |
| | | Hapten-carrier protein | |
| | | conjugation | |
| Sulfo- | Primary amines | Stable maleimide reactive | 11.6 A |
| SMCC | Sulfhydryls | group | |
| | | Water-soluble | |
| | | Enzyme-antibody | |
| | | conjugation | |
| MBS | Primary amines | Enzyme-antibody | 9.9 A |
| | Sulfhydryls | conjugation | |
| | | Hapten-carrier protein | |
| | | conjugation | |
| Sulfo- | Primary amines | Water-soluble | 9.9 A |
| MBS | Sulfhydryls | | |
| SIAB | Primary amines | Enzyme-antibody | 10.6 A |
| | Sulfhydryls | conjugation | |
| Sulfo- | Primary amines | Water-soluble | 10.6 A |
| SIAB | Sulfhydryls | | |
| SMPB | Primary amines | Extended spacer arm | 14.5 A |
| | Sulfhydryls | Enzyme-antibody | |
| | | conjugation | |
| Sulfo- | Primary amines | Extended spacer arm | 14.5 A |
| SMPB | Sulfhydryls | Water-soluble | |
| EDC/Sulf | Primary amines | Hapten-Carrier | 0 |
| o-NHS | Carboxyl groups | conjugation | |
| ABH | Carbohydrates | Reacts with sugar groups | 11.9 A |
| | Nonselective | | |

It has been shown that surface-bound targeting ligands can be shielded by other particle components or surface-adsorbed species (Allen et al., 1995). However, recent research has shown that when targeting ligands are tethered to surface-bound spacers, the probability of these ligands to capture binding sites and the distance at which site recognition occurs are both increased as the tether length is increased (Jeppesen et al., 2001).

II. Nanocapsules

In certain aspects, the invention provides micellar nanocapsules targeted to the systemic skeleton. By forming such nanoparticles from amphipathic materials with a very low critical micelle concentration (CMC), micellar nanoparticles may be produced that are stable under physiological conditions. Bioactive payloads, such as agents that heal fractures, treat metastatic lesions, prevent bone loss, build bone tissue etc., may be enclosed in these structures. Particular benefit will be obtained for delivery of hydrophobic agents in accordance with the invention.

The term “nanoparticle” as used herein includes particles ranging in size from several nanometers to several micrometers in diameter. A “nanocapsule” refers to a nanoparticle encapsulating one or more agents. One important aspect of nanoparticle formation and associated therapies is the choice of the starting components to achieve vesicle stability and the improvement in circulation half-life in vivo. Certain PEGylated phospholipid conjugates may in certain embodiments find use for the formation of nanoparticles. See Allen, et al. (1991). DSPE-PEG in particular may find use with the invention. Phospholipids that are PEGylated could potentially be from natural sources, such as egg or soybean phosphatidylcholine, brain phosphatidic acid, brain or plant phosphatidylinositol, heart cardiolipin and plant or bacterial phosphatidylethanolamine could be used, although are generally preferably not used as the primary phosphatide, i.e., constituting 50% or more of the total phosphatide composition, because of the instability and leakiness of the resulting particle. In particular embodiments, a bioactive factor may be, for example, encapsulated in the hydrophobic interior of a nanoparticle provided by the invention.

Micellar nanoparticles may be formed by mixing appropriate starting amphipathic materials as described herein in an aqueous environment above the CMC in a container, for example, a glass, pear-shaped flask. Hydrophobic bioactive agents may be admixed with the amphipathic material during micellar nanoparticle formation to achieve encapsulation. During the process it may be beneficial for the container to have a volume several-times greater than the volume of the expected suspension of nanoparticles. Using a rotary evaporator, solvents can be removed at approximately 40° C. under negative pressure.

In certain aspect nanoparticle compositions may be dried for storage. Dried particles can be hydrated at approximately 25-50 mM in sterile, pyrogen-free water by shaking until the film is resuspended. The aqueous particles can be then separated into aliquots, each placed in a vial, lyophilized and sealed under vacuum.

Unencapsulated additional materials, such as agents including but not limited to hormones, drugs and the like, may be removed by centrifugation or by size exclusion chromatography. The purified nanoparticles may be resuspended at an appropriate total micelle concentration. The amount of additional material or active agent encapsulated can be determined in accordance with standard methods. After determination of the amount of additional material or active agent encapsulated in the preparation, the nanoparticles may be diluted to appropriate concentrations and stored at 4° C. until use. A pharmaceutical composition comprising the nanoparticles will usually include a sterile, pharmaceutically acceptable carrier or diluent, such as water or saline solution.

The size of a nanoparticle varies depending on the method of synthesis. Nanoparticles in the present invention can be a variety of sizes. In accordance with the invention, it will be desired in certain embodiments that nanocapsules are sufficiently small to cross the blood vessel wall. Such nanocapsules will generally have a size of about 100 nm or smaller, including about 90 nm, about 80 nm, about 70 nm, about 60 nm, or less than about 50 nm in external diameter. In preparing such nanocapsules, any protocol described herein, or as would be known to one of ordinary skill in the art may be used.

A micellar nanoparticle suspended in an aqueous solution is generally in the shape of a spherical vesicle, having a surface arranged such that the hydrophilic moieties tend to remain in contact with an aqueous phase and the hydrophobic regions tend to self-associate in the core. Aggregates may form when the hydrophilic and hydrophobic parts of more than one micellar nanocapsule become associated with each other. The size and shape of these aggregates will depend upon many different variables, such as the nature of the solvent and the presence of other compounds in the solution.

The production of micellar nanocapsules can be accomplished, for example, by sonication of starting amphipathic material such as PEGylated phospholipids. In one aspect, a contemplated method for preparing nanocapsules is heating sonicating, and sequential extrusion through filters or membranes of decreasing pore size, thereby resulting in the formation of small, stable nanoparticles. The most common pieces of instrumentation for preparation of sonicated particles are bath and probe tip sonicators. Cup-horn sonicators may also be used. Probe tip sonicators deliver high energy input to suspensions but can suffer from overheating causing degradation. Sonication tips also tend to release titanium particles into the lipid suspension which must be removed by centrifugation prior to use. For these reasons, bath sonicators may be preferred.

Sonication of a solution may be accomplished by placing a test tube containing the sample in a bath sonicator (or placing the tip of the sonicator in the test tube) and sonicating for 5-10 minutes. Mean size and distribution is influenced by composition and concentration, temperature, sonication time and power, volume, and sonicator tuning. Since it is nearly impossible to reproduce the conditions of sonication, size variation between batches produced at different times is not uncommon.

Extrusion can also be used to size particles. Extrusion is a technique in which a suspension is forced through a polycarbonate filter with a defined pore size to yield particles having a diameter near the pore size of the filter used. Prior to extrusion through the final pore size, a suspension can be disrupted either by several freeze-thaw cycles or by prefiltering the suspension through a larger pore size (typically 0.2 μm-0.1 μm). This method helps prevent membranes from fouling and improves the homogeneity of the size distribution of the final suspension. As with all procedures for downsizing particle dispersions, the extrusions are generally performed at a temperature above the Tc of any lipids used. Attempts to extrude below the Tc can foul with rigid membranes which cannot pass through the pores. Extrusion through filters with 100 nm pores typically yields a mean particle diameter of 120-140 nm.

Once manufactured, nanoparticles can be used to deliver encapsulated bioactive factors for targeting to bone, as is described herein. Nanoparticles can interact with cells to deliver agents via at least four different mechanisms: Endocytosis by phagocytic cells of the reticuloendothelial system such as macrophages and/or neutrophils; adsorption to the cell surface, either by nonspecific weak hydrophobic and/or electrostatic forces, and/or by specific interactions with cell-surface components; fusion with the plasma cell membrane by insertion of the surface of the nanoparticle into the plasma membrane, with simultaneous release of the contents into the cytoplasm; and/or by transfer of nanoparticle lipids to cellular and/or subcellular membranes, and/or vice versa, without any association of the nanoparticle contents. Varying the nanoparticle formulation can alter which mechanism is operative, although more than one may operate at the same time.

B. Timed or Triggered Release of Nanocapsule Payloads

In certain embodiments of the invention, the use of nanocapsules designed for sustained, triggered or timed release is contemplated. For example, nanocapsules may be designed to release payloads of bioactive factors upon contact with a given signal, for example, that is released by bone. In this way, nanocapsule payloads are targeted not only to bone but delivered to bone in need of treatment with the given bioactive factor. Such a signal could be endogenous or externally administered. An external signal could be used to cause release of the nanocapsule payloads by, for example, using a chemical signal or physical signal. Examples of physical signals include administration of ultrasound or heat. In this manner the signal could be administered only to the site where treatment with the bioactive factor is needed, maximizing delivery of the factor to the site where needed and minimizing exposure to other parts of the body. Sustained release nanocapsule formulations could also be used. In this manner the efficacy of treatment may be maximized by maintaining therapeutic levels of the bioactive factor over time, without the need for continual administrations of the nanocapsules.

Temporally pulsed release of nanocapsules is also specifically contemplated. This could be achieved, for example, by administration of several types of nanocapsules having different delayed release characteristics. Such temporally pulsed techniques may yield benefits beyond those available with sustained release formulations. For example, increased bone matrix generation activity is observed in systems subjected to periodic exposure to bioactive factors in contrast to systems subjected to sustained exposure to the bioactive factor. This increased matrix generation may be due to the unhindered completion of the natural matrix generation cascade triggered by a spiked dosage of bioactive factor at the site. Also, sustained release may cause a physiologic acclimation that suppresses the triggered response to the spiked elevations in the bone factor concentration.

C. Kits

Nanocapsules prepared in accordance with the invention may be comprised in a kit. In a non-limiting example, nanocapsules targeted to bone and containing payloads comprising one or more bioactive factors may be comprised in a kit. The kits will thus comprise, in suitable container means, nanocapsules of the present invention.

The kits may comprise the nanocapsules in a suitably aliquoted formulation. The components of the kits may be packaged in aqueous media or in lyophilized form. The container means of the kits will generally include at least one vial, test tube, flask, bottle, syringe or other container means, into which a component may be placed, and preferably, suitably aliquoted. Where there are more than one components in the kit, the kit also will generally contain a second, third or other additional container into which the additional components may be separately placed. However, various combinations of components may be comprised in a vial. The kits of the present invention also will typically include a means for containing the nanocapsules and any other reagent containers in close confinement for commercial sale. Such containers may include injection or blow-molded plastic containers into which the desired vials are retained.

Therapeutic kits of the present invention may include pharmaceutical compositions for delivery of bioactive factor-containing nanocapsules targeted to bone. Such kits will generally contain, in suitable container means, a pharmaceutically acceptable formulation of nanocapsules. The kit may have a single container means, and/or it may have distinct container means for each compound.

When the components of the kit are provided in one and/or more liquid solutions, the liquid solution is an aqueous solution, with a sterile aqueous solution being particularly preferred. The nanocapsule compositions may also be formulated into a syringeable composition. In which case, the container means may itself be a syringe, pipette, and/or other such like apparatus, from which the formulation may be injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

Irrespective of the number and/or type of containers, the kits of the invention may also comprise, and/or be packaged with, an instrument for assisting with the injection/administration and/or placement of the ultimate nanocapsule containing composition within the body of an animal. Such an instrument may be a syringe, pipette, forceps, and/or any such medically approved delivery vehicle.

III. Pharmaceutical Compositions

In certain aspects of the current invention, pharmaceutical compositions are provided for delivering nanocapsules containing bioactive factors to patients or subjects in need thereof. Pharmaceutical compositions of the present invention thus comprise an effective amount of one or more bioactive factors contained in nanocapsules in addition to any other desired components dissolved or dispersed in a pharmaceutically acceptable carrier.

An “effective amount” is the amount of an bioactive or therapeutic compound, agent or factor that is sufficient to treat or prevent a bone related condition or disease associated in a patient or subject. Thus and “effective amount” is one that preferably reduces the amount of symptoms of the condition in the infected patient by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems such as those described in the examples or any of those known to one of skill in the art.

The phrases “pharmaceutical or pharmacologically acceptable” refers to molecular entities and compositions that do not produce an adverse, allergic or other untoward reaction when administered to an animal, such as, for example, a human, as appropriate. The term “bioactive factor” or “is intended to refer to a chemical entity, whether in the solid, liquid, or gaseous phase which is capable of providing a desired therapeutic effect when administered to a subject in accordance with the invention. The term “bioactive factor” includes synthetic compounds, natural products and macromolecular entities such as polypeptides, polynucleotides, or lipids and also small entities such as neurotransmitters, ligands, hormones or elemental compounds. The term also includes such compounds whether in a crude mixture or purified and isolated.

The preparation of a pharmaceutical composition that contains at least one nanocapsule or other ingredients will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the nanocapsules of bioactive factors, its use in the therapeutic or pharmaceutical compositions is contemplated.

The nanocapsule-containing pharmaceutical composition may be comprised in different types of carriers depending on whether it is to be administered in solid, liquid or aerosol form, and whether it need to be sterile for such routes of administration as injection. The nanocapsules can potentially be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g. aerosol inhalation), injection, infusion, continuous infusion, localized perfusion bathing target cells directly, via a catheter, via a lavage, in cremes, in lipid compositions, or by other method or any combination of the forgoing as would be known to one of ordinary skill in the art (see, for example, Remington's Pharmaceutical Sciences, 18th Ed. Mack Printing Company, 1990, incorporated herein by reference). In certain aspects of the invention, non-invasive administration techniques in particular may be used advantageously, for example, intranasal administration.

The actual dosage amount of a pharmaceutical composition of the present invention administered to a patient can be determined by physical and physiological factors such as body weight, severity of condition, the type of disease being treated, previous or concurrent therapeutic interventions, idiopathy of the patient and on the route of administration. This amount may also be adjusted based on the targeting agent used for the nanocapsules. One advance of the current invention is that targeting allows usage of doses lower than required using non-targeted treatments.

The practitioner responsible for administration will, in any event, determine the concentration of bioactive factor(s) and nanocapsules in a composition and appropriate dose(s) for the individual subject. In certain embodiments, pharmaceutical compositions may comprise, for example, an overall concentration of at least about 0.1% of an active compound, including, for example, about 0.1% to about 75%, 0.1% to about 50%, 0.1% to about 25%, 0.1% to about 10%, 0.1% to about 5%, 0.1% to about 3%, 0.1% to about 1%, 1% to about 10% and about 5% to about 15%.

In other non-limiting examples, a dose may also comprise, in nanocapsule carriers, about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, and about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered in nanocapsule payloads, based on the numbers described above.

In addition to nanocapsules, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The bioactive factor that is used may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the nanocapsule composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. It will be necessary that such a carrier does not disrupt the nanocapsules prior to delivery to a patient. The proper fluidity of the composition can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In some cases, it will be preferable to include isotonic agents, such as, for example, sugars, sodium chloride or combinations thereof.

In other embodiments of the invention, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the nanocapsules are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain further embodiments an oral composition may comprise one or more binders, excipients, disintegration agents, lubricants, flavoring agents, and combinations thereof. Such a composition may comprise, for example, one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating bioactive factors, e.g., in nanocapsules, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Targeting Ligand Synthesis and Conjugation to Phospholipids

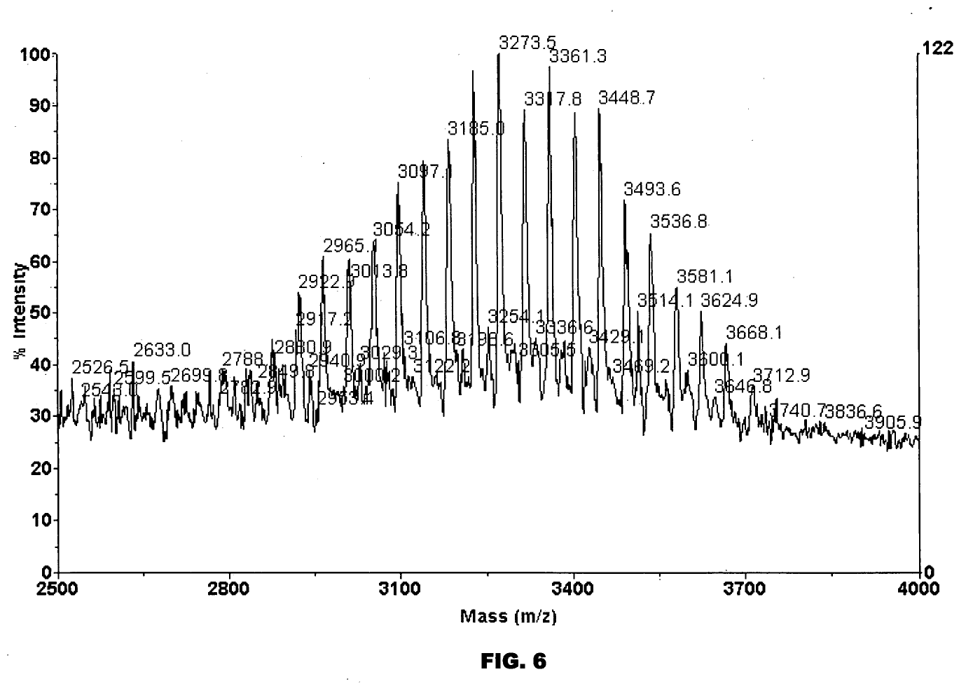

The amino-functionalized analogue of MBP was synthesized by known methods (Uludag et al., Biotechnol. Prog., 16, 258-267 (2000)) and purified by chromatography. A six mer oligomer of aspartic acid (ASP₆) was custom synthesized (New England Peptide, Inc.). The N-terminus of both ligands was converted to thiol with 2-iminothiolane (2-IT) and the products subsequently conjugated to maleimide functionalized distearoyl phosphotidylethanolamine-N-methoxypolyethylene glycol-2000 (DSPE-PEG₂₀₀₀-M, Northern Lipids). The conjugate structures were analyzed by MALDI-TOF/MS (FIG. 6). The MALDI spectrum for neat DSPE-PEG₂₀₀₀-M, indicated it has a median mass of about 3010. Conjugation of this functionalized lipid to the thiolated MBP ligand (FW=280.2) shifted the median mass accordingly, yielding conjugate with a median mass of about 3305. Conjugation of the Asp₆ ligand to the same lipid was confirmed similarly.

### Example 2

### In Vitro Targeting of Ligand-Phospholipid Conjugates

The two ligands a-MBP and Asp₄, were separately conjugated to fluorescein isothiocyanate (FITC) through their N-terminus. The ligands were each dissolved in HEPES buffer and FITC added in 2× molar excess. The reaction proceeded for 24 hours, after which, the product was dialyzed to remove unreacted components. Table 1 lists the properties of four different hydroxyapatite powders used to study the extent of labeled ligand adsorption. Scanning electron micrographs were prepared of each of the powders. The powders were washed with phosphate buffer solution and dried before use. The fluorescein-labeled ligands were mixed with varying quantities of the HAp powders to produce dispersions containing from 0 to 100 nmol of ligand/mg of HAp substrate. The dispersions were incubated at room temperature for 24 hours, centrifuged, the particulate-free liquid collected, and analyzed FITC content by UV-Vis at 510 nm. Unconjugated FITC in HEPES buffer was used as a control.

TABLE 1
Hydroxyapatite powders used in targeting studies

| Name | Source | BET Surface Area (m²/g) |
|---|---|---|
| HA-1 | BioForm, Inc. | 0.22 ± 0.03 |
| HA-2 | Prof. Ong, UTHSC | 16.07 ± 0.95 |
| HA-3 | Prof. Bandy, WSU | 29.67 ± 2.15 |
| HA-4 | BioRad | 863 ± 56 |

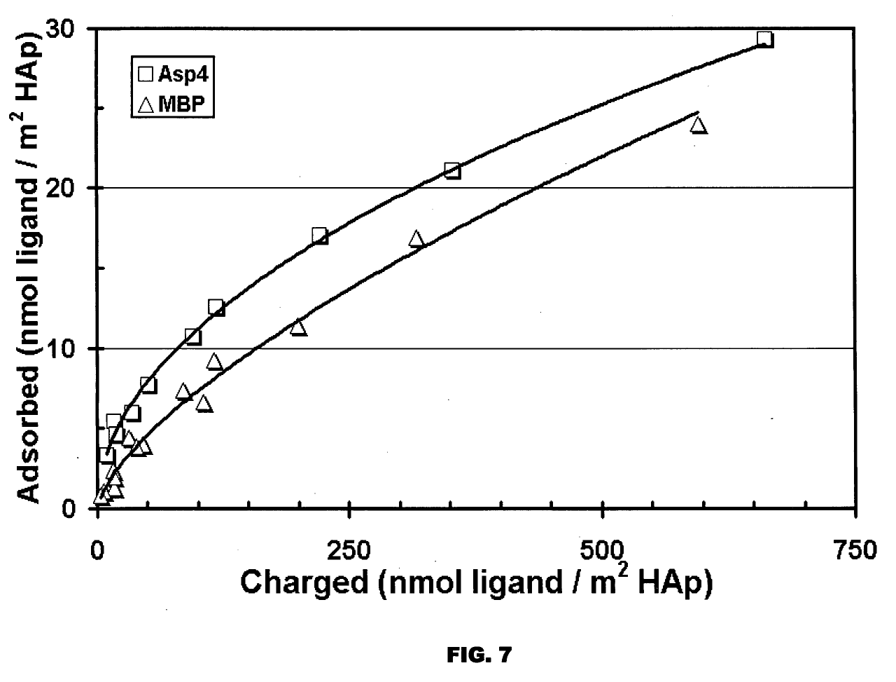

FIG. 7 shows the surface area normalized adsorption of FITC-labeled ligands onto the various HAp substrates. Adsorption of neat FITC onto each of the substrates was insignificant. The adsorption of the ligands onto HA-1 was insignificant relative to the other substrates. Ligand adsorption increased with available surface area of the substrate and with increasing amounts of ligand charged to the system. Both ligands appear to have similar affinities for the HAp substrates. These curves suggest the Asp₄ ligand adsorbs slightly better than the MBP ligand

### Example 3

### Preparation of Bone-Targeting Liposomes

Liposomes were prepared from distearoyl-phosphatidylcholine, cholesterol, α-tocopherol, and DSPE-PEG₂₀₀₀ in the molar ratios 1:1:0.04:0.05, respectively, by hydration of lyophilized lipid films followed by sizing through nanoporous filters and purification (www.avantilipids.com; Mayer et al., 1986). Particle size and distribution were analyzed (N4 Plus, Beckman-Coulter) to confirm target particle size of the final liposomes.

Ligand-phospholipid conjugates were inserted into preformed liposomes using the method of post-insertion (Uster et al., 1996). Briefly, micelles of ligand-phospholipid conjugates were prepared by sonicating a quantity of material in HEPES buffer to obtain a dispersion. The micelles were incubated with preformed liposomes at 60° C. for approximately one-hour, after which the liposomes were separated by size exclusion chromatography. The ligand content of the modified liposomes was determined by complexing the ligand with ^{99m}Tc and performing liquid scintillation counting.

### Example 4

### In Vitro Targeting to Hydroxyapatite of Bone-Targeting Liposomes

Bone-targeting liposomes were prepared as described in Example 2. Only liposomes containing MBP ligands were studied in this example. Liposomes were charged to a well and a hydroxyapatite dispersion added to produce suspension containing a known amount of ligand per unit of hydroxyapatite. The suspension was incubated with agitation at 37° C. for one hour, after which, the wells were charged with a known amount of ^{99m}Tc to chelate free ligands. The wells were incubated for 30 minutes, and the well contents removed, centrifuged, and separated into liquid and solid fractions, which were then each subjected to gamma counting.

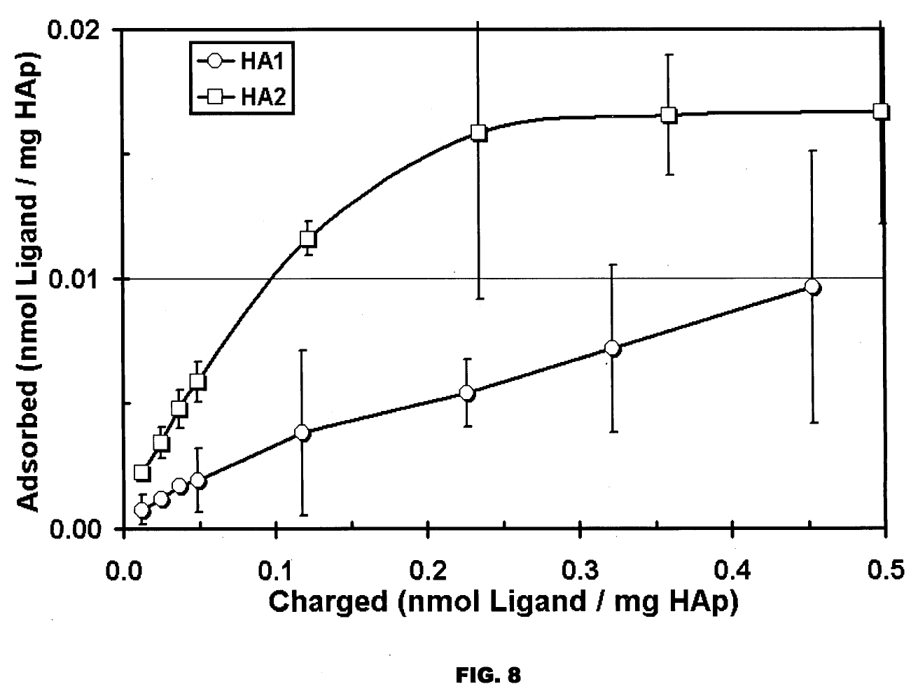

FIG. 8 shows the relative adsorption MBP ligand-containing liposomes onto the HAp substrates. Liposomes with no MBP ligand did not target the substrates. The extent of liposome targeting did increase with available surface area. However, normalization of the adsorption data by the available surface area did not produce a single curve. This was probably due to the size of the liposomes relative to the size of the substrate powders and their ability to penetrate between adjacent particles.

### Example 5

### Preparation of Bone-Targeting Micelles

Micelles of MBP-phospholipid conjugates were prepared by a phase separation technique. Briefly, the lipid is dissolved in a small quantity of chloroform and the solution emulsified in HEPES buffer. The emulsion is sonicated at 60° C., yielding a clear, colorless liquid. The particle size distribution of methylene bisphosphonate-phospholipid conjugate micelles gave a mean of 21.4±5.6 nm, which was typical of micelles. The measured micelle size was in good agreement with the predicted particle size of approximately 30 nm.

### Example 6

### Encapsulation of Lovastatin in Bone-Targeting Micelles

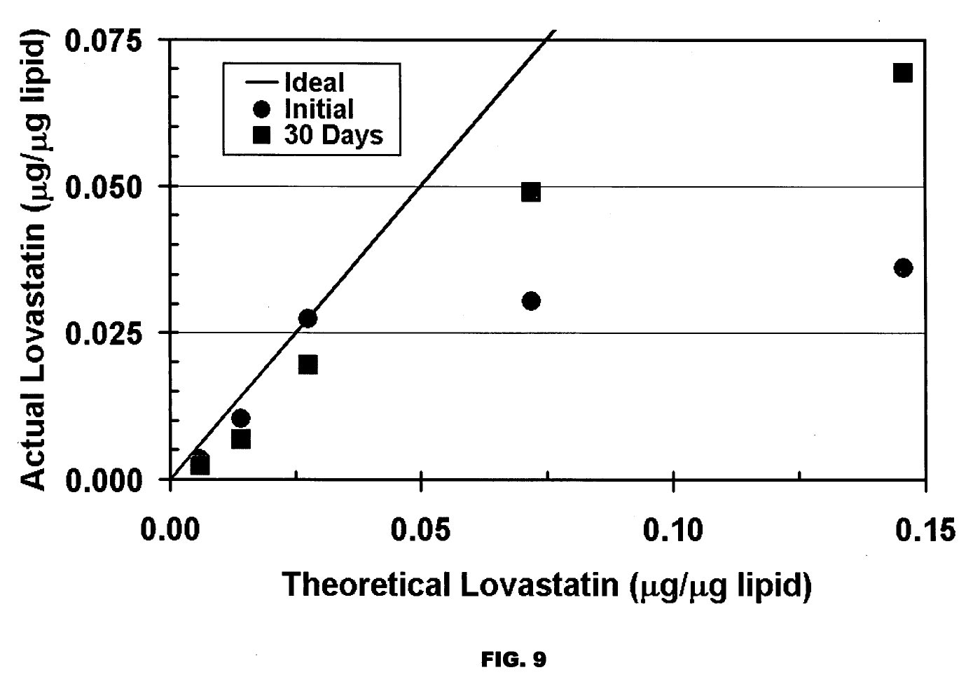

Lovastatin was encapsulated in MBP-lipid conjugate micelles at ratios ranging from about 1:1 to 1:50 mol:mol lovastatin:lipid using the phase separation technique. All preparations initially produced clear, colorless solutions, indicating complete solubilization of lovastatin by the lipid. However, over time, usually within about 24 hours, needle-like crystals were observed on the surface of the fluid. Comparison of the crystals with lovastatin crystallized from chloroform in HEPES buffer suggested they were lovastatin. The crystals were confirmed to be lovastatin by HPLC analysis. FIG. 9 shows the lovastatin content of the micelle solutions as determined HPLC. The data indicate lovastatin was stabilized in micelles up to about 0.03 μg/μg of lipid (˜1:5 mol:mol lovastatin:lipid), in excellent agreement with solubility values predicted from simulation. However, if the solutions were allowed to age, then the amount of stabilized lovastatin increased slightly over all compositions. Particle size analysis of the aged compositions showed an increase in the micelle particle size, suggesting the increased lovastatin content is concomitant with the particle size increase.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,013,649
U.S. Pat. No. 5,401,511
U.S. Pat. No. 5,603,872
U.S. Pat. No. 5,889,155
U.S. Ser. No. 10/350,805
Adams Invest New Drugs, 18:109-121, 2000.
Allen et al., Biochim. Biophys. Acta, 1066:29-36, 1991.
Allen et al., Biochim. Biophys. Acta, 1237:99-108, 1995.
Baldwin et al., Med. Sci. Sports Exerc., 10:1247-1253, 1996.
Bataille et al., N Eng J Med, 336:1657-1664, 1997.
Bikle et al., J. Bone Miner. Res., 9:1777-1787, 1994a.
Bikle et al., J. Bone Miner. Res., 9:1789-1796, 1994b.
Carmeliet et al., Bone, 22:139S-143S, 1998.
Centrella, et al. J. Biol. Chem., 262:2869-2874, 1987.
Chenu, et al. Proc. Natl. Acad. Sci. USA, 85:683-5687, 1988.
Cooper et al., Quarterly J. Med., 87:203-209, 1994.
Cronhjort et al., Acta Radiol., 40:309-311, 1999.
Davis and Jones, Semin. Nucl. Med., 6:19-31, 1976.
Fleisch, Bisphosphonates in Bone Disease, 2^{nd} Edition, Pantheon, New York, 1995.
Forssen and Willis, Adv. Drug Delivery Reviews, 29:249-271, 1998.
Fujisaki et al., J. Drug Targeting, 3:273-282, 1995.
Fujisaki et al., J. Pharm. Pharmacol., 48:798-800, 1996.
Garrett et al., J. Clin. Invest., 111:1771-1782 (2003).
Halloran et al., J. Bone Miner. Res., 10:1168-1176, 1995.
Harris et al., Bone, 26:325-331, 2000.
Holick, The Lancet, 355:1607-1611, 2000.
Jemal et al., CA Cancer J Clin, 52:23-45, 2002.
Jeppesen et al., Science, 293:465-468, 2001.
Joseph, Am. J. Hosp. Pharm., 51:188-197, 1994.
Kamps et al., Proc. Natl. Acad. Sci., USA, 94:11681-11685, 1997.
Kasugai et al., J. Bone and Mineral Research, 15:936-943, 2000.
Kontoci et al., Synth. Comm., 26:2037-2043, 1996.
Kyle et al., Mayo Clin Proc, 78:21-33, 2003.
Lantto et al., Acta Radiol., 28:631-633, 1987.
Lee and Huang, Biochim. Biophys. Acta, 1233:134-144, 1995.
Machwate et al., J. Clin. Invest., 96:1245-1253, 1995.
Martin et al., J. Clin. Invest., 261, 256-26, 1978.
Mayer et al., Biochim. Biophys. Acta, 858, 161-168, 1986.
McCarthy et al., Eur. J. Clin. Invest., 30:1044-1054, 2000. Mundy, Adv. Drug Delivery Rev., 42:165-173, 2000.
Mundy, In: Primer on the metabolic bone diseases and disorders of mineral metabolism, Favus, (Ed.) Amer. Soc. of Bone and Mineral Research, 16-24, 1996.
Mundy et al., Science, 286:1946-1949, 1999.
Mundy, Eur. J. Cancer, 34:246-251, 1998.
Nagata et al., Biochem. J., 274(Pt. 2):513-520, 1991.
National Geographic, January, 2001.
Parfitt et al., J. Bone Miner. Res., 11:150-159, 1996.
Parkin et al., CA Cancer J Clin, 49:33-64, 1999.
Remington's Pharmaceutical Sciences, 18th Ed. Mack Printing Company, 1990.
Rho et al., Medical Engineering and Physics, 20, 1998.
Senior and Gregoriadis, Life Sci., 30:2123-2136, 1982.
ten Dijke et al., Bio/Technology, 7:793-798, 1989.
Trends in Orthopedics, ABM AMRO, April, 2000.
Turner, J. Appl. Physiol., 89:840-847, 2000.
Uludag et al., Biotechnol. Prog., 16:1115-1118, 2000a.
Uludag et al., Biotechnol. Prog., 16:258-267, 2000b.
Uster et al., FEBS Letters, 386, 243-246, 1996.
Vico et al., The Lancet, 355:1607-1611, 2000.
Westerlind and Turner, J. Bone Miner. Res., 10:843-848, 1995.
Wu et al., Cancer Res., 53:3765-3770, 1993.
Yokogawa et al., Endocrinology, 142(3):1228-1233, 2000.
Zerath et al., J. Appl. Physiol., 79:1889-1894, 1995.

1. A nanocapsule comprised of an amphipathic surface encapsulating at least a first hydrophobic bioactive factor, wherein the surface comprises at least a first ligand having affinity for a component of the systemic skeleton.

2. The nanocapsule of claim 1, defined as a micellar nanocapsule.

3. The nanocapsule of claim 1, wherein said ligand has affinity for hydroxyapatite in said systemic skeleton.

4. The nanocapsule of claim 1, wherein said ligand is a bisphosphonate comprising the structure: 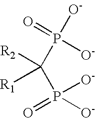
wherein R1 is H, OH or, Cl and wherein R2 is an alkyl amine or other heterobifunctional linker coupled to the nanocapsule surface.

5. The nanocapsule of claim 1, wherein said ligand is amino methylene bisphosphonate (aMBP).

6. The nanocapsule of claim 1, wherein said ligand is a protein, a peptide an oligopeptide, or an antibody.

7. The nanocapsule of claim 6, wherein said protein or peptide comprises an oligopeptide comprising the sequence Aspₙ.

8. The nanocapsule of claim 1, wherein n=6.

9. The nanocapsule of claim 6, wherein said protein or peptide comprises the sequence of osteopontin or bone sialoprotein or a fragment thereof.

10. The nanocapsule of claim 1, wherein said nanocapsule has a diameter of from 1 nm to 200 nm.

11. The nanocapsule of claim 10, wherein said nanocapsule has a diameter of from about 50 nm to about 100 nm.

12. The nanocapsule of claim 10, wherein said nanocapsule has a diameter of from about 100 nm to about 150 nm.

13. The nanocapsule of claim 10, wherein said nanocapsule has a diameter of from about 150 nm to about 200 nm.

14. The nanocapsule of claim 1, wherein the first target ligand is covalently bound to said surface.

15. The nanocapsule of claim 1, wherein the bioactive factor is an osteotropic agent.

16. The nanocapsule of claim 1, wherein the bioactive factor is an anabolic agent.

17. The nanocapsule of claim 1, wherein the amphipathic surface is comprised of a block polymer.

18. The nanocapsule of claim 1, wherein the amphipathic surface is comprised of polyethylene glycol-modified phospholipid.

19. The nanocapsule of claim 18, wherein the amphipathic surface is comprised of distearyol phosphoethanolamine-N-polyethylene glycol (DSPE-PEG).

20. The nanocapsule of claim 1, wherein the amphipathic surface is comprised of a material with a critical micelle concentration of between about 1 μm and about 20 μm.

21. A method of delivering a bioactive factor to a component of the systemic skeleton of a patient in need thereof comprising:
(a) obtaining nanocapsules comprised of an amphipathic surface encapsulating at least a first hydrophobic bioactive factor, wherein the surface comprises at least a first ligand having affinity for a component of the systemic skeleton; and
(b) administering the composition to the patient.

22. The method of claim 21, wherein the nanocapsules are micellar nanocapsules.

23. The method of claim 21, wherein said ligand has an affinity for hydroxyapatite in said systemic skeleton.

24. The method of claim 21, wherein the ligand is amino methylene bisphosphonate (aMBP).

25. The method of claim 21, wherein the ligand comprises an oligopeptide that has the following molecular formula:

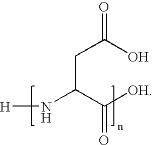

26. The method of claim 21, wherein said composition further comprises a pharmaceutically acceptable carrier.

27. The method of claim 21, wherein said bioactive factor is released from said nanocapsules upon contact of said nanocapsule with a signal released from said systemic skeleton.

28. The method of claim 21, wherein said nanocapsules comprise varied temporal release characteristics.

29. The method of claim 21, wherein said nanocapsules are from about 1 nm to about 200 nm in diameter.

30. The method of claim 21, wherein said nanocapsules comprise a mean diameter of from about 50 nm to about 100 nm.

31. The method of claim 21, wherein said nanocapsules comprise a mean diameter of from about 100 nm to about 150 nm.

32. The method of claim 21, wherein said nanocapsules comprise a mean diameter of from about 150 nm to about 200 nm.

33. The method of claim 21, wherein the first target ligand is covalently bound to said surface.

34. The method of claim 21, wherein said bioactive factor is selected from the group consisting of a statin, a proteasome inhibitor, and an antiosteoporotic alkyloid.

35. The method of claim 21, wherein said bioactive factor is an osteotropic agent.

36. The method of claim 21, wherein said bioactive factor is hormonally active.

37. The method of claim 21, wherein the bioactive factor is an anabolic agent.

38. The method of claim 21, wherein the amphipathic surface is comprised of a block polymer.

39. The method of claim 21, wherein the amphipathic surface is comprised of polyethylene glycol-modified phospholipid.

40. The method of claim 21, wherein the amphipathic surface is comprised of distearyol phosphoethanolamine-N-polyethylene glycol (DSPE-PEG).

41. The method of claim 21, wherein the amphipathic surface is comprised of a material with a critical micelle concentration of between about 1 μm and about 20 μm.

42. The method of claim 21, wherein said composition is administered locally, systemically, intravenously, intra-arterially, topically or orally.

43. A method of preventing bone loss in a subject in need thereof comprising:
(a) (Currently amended) obtaining nanocapsules comprised of an amphipathic surface encapsulating at least a first hydrophobic osteotropic factor, wherein the surface comprises at least a first ligand having affinity for a component of the systemic skeleton; and
(b) administering the composition to the patient.

44. The method of claim 43, wherein the nanocapsules are micellar nanocapsules.

45. The method of claim 43, wherein said ligand has an affinity for hydroxyapatite in said systemic skeleton.

46. The method of claim 43, wherein said osteotropic factor is released from said nanocapsules upon contact of said nanocapsules with a signal released from said systemic skeleton.

47. The method of claim 43, wherein the nanocapsules comprise varied release characteristics.

48. The method of claim 43, wherein the ligand is amino methylene bisphosphonate (aMBP).

49. The method of claim 43, wherein the ligand is an oligopeptide that has the following molecular formula:

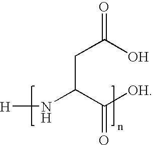

50. The method of claim 43, wherein said composition further comprises a pharmaceutically acceptable carrier.

51. The method of claim 43, wherein said composition is administered locally, intranasally, systemically, intravenously, intra-arterially, topically or orally.
